(19) United States
(12) Patent Application Publication
(10) Pub. No.: US 2017/0035982 A1
(43) Pub. Date: Feb. 9, 2017

(54) SMALL ANIMAL IMAGING DEVICE

(71) Applicant: Jack G. Roseman, Philadelphia, PA (US)

(72) Inventor: Jack G. Roseman, Philadelphia, PA (US)

(21) Appl. No.: 15/288,859

(22) Filed: Oct. 7, 2016

(51) Int. Cl. A61M 16/06; A61B 6/00; A61B 5/055; A61B 6/03; A61M 16/01; A61M 16/00

(52) U.S. Cl. CPC A61M 16/0683; A61M 16/01; A61M 16/0093; A61B 5/055; A61B 6/032; A61B 6/037; A61B 6/508; A61M 2250/00; A61B 2503/40

(57) ABSTRACT

An anesthesia mask configured to be worn on an animal's head. The mask includes a contoured surface that substantially follows the contours of a portion of the animal's head and defines a cavity that at least partially receives the head. One or more outlets in communication with the nose and/or the mouth are formed in the contoured surface. The mask includes a first opening configured to receive anesthesia and at least one channel that conducts the anesthesia from the first opening to the outlet(s). The mask may include an airflow collection system that includes inlet(s) formed in the contoured surface, a second opening connectable to a vacuum device, and passageway(s) configured to conduct an unused portion of the anesthesia from the inlet(s) to the second opening. The mask may include a body portion and a removable bite bar component that include first and second portions, respectively, of the contoured surface.

### BACKGROUND OF THE INVENTION

Field of the Invention

The present invention is directed generally to masks used to deliver anesthesia and more particularly to masks used to anesthetize animals (e.g., mice and rats).

Description of the Related Art

Gas is used to anesthetize rodents and other small animals during imaging (e.g., fully body scans) to minimize body motion and capture higher quality scans with fewer artifacts caused by unwanted motion. Standard protocols for imaging such animals for cancer research include anesthetization using an anesthesia mask compatible with the particular scanning technology being used. Unfortunately, currently available anesthesia masks used by research facilities are too simple to handle dangerous gas(es), or too complex and not user-friendly.

Currently available masks utilize a bite bar mechanism to fix a rodent's head inside a cylindrically shaped or cone shaped chamber that approximates the largest dimension of the head, allowing complete coverage of the head up to the ears, and usually include ear pins that are steaked into the animal's ear canals. Unfortunately, such masks have many problems and limitations. For example, while this design can add greater head stability, a slight external force can knock the animal's incisors out of the bite bar mechanism, allowing the head to fall out of alignment with the scanning machine. Additionally, this design is only satisfactory for imaging rodents in a prone position because the design relies on gravity to keep the head fixed on the bite bar mechanism and the incisors hooked onto the bite bar mechanism. In the prone position, normal respiration causes chest and underside contractions that can cause unwanted head movement, resulting in motion artifacts in the images and, thus, lower quality scans.

Respiration-induced motion may be remedied by repositioning the animal, usually in a supine position, where chest and underside motion does not physically interfere with the rest of the body or scanning region. Unfortunately, time is often wasted trying to place the animal in position where the animal is completely still and remains unaffected by external forces. When the animal is placed in a supine position, many existing masks (designed for prone imaging) will typically be used in an upside-down orientation to achieve higher quality scans.

Further, as a result of their simple construction, currently available masks are not made to handle dangerous gas(es). In fact, it is inaccurate to refer to them as being “gas masks” because many of them do not deliver gas correctly, efficiently, or safely. In many current designs, using the aforementioned positioning mechanism, the animal's head is suspended in the mask, leaving a void in which dangerous gas(es) may collect before being inhaled. Most existing masks are not configured to collect and remove unused gas, because such a mechanism is too complex, which may allow dangerous anesthesia gas(es) to leak into the environment. This also reduces the efficacy of gas delivery.

More complex and expensive gas-delivery apparatuses that use a valved system to deliver and collect gas are available and solve the problem of anesthesia gas(es) leaking into the environment. Unfortunately, the valves in these designs rely on the misconception that all mice breathe nasally, which renders such designs useless for experimental animals that breathe orally. While the more advanced apparatuses are better than the simpler designs in terms of head stability, they similarly do not give users full autonomy to change the position of the animal (e.g., from prone to supine and vice versa) to limit respiration-induced motion.

Thus, a need exists for new anesthesia masks for use by animals. Animal anesthesia masks that include a system for collecting gas are particularly desirable. An animal anesthesia mask configured to function in whichever position (e.g., prone or supine) the animal is positioned is also desirable. The present application provides these and other advantages as will be apparent from the following detailed description and accompanying figures.

### BRIEF DESCRIPTION OF THE SEVERAL VIEWS OF THE DRAWING(S)

FIG. 1A is a side view of a Small Animal Imaging Device (“SAID”) illustrated worn on a head of an animal in a prone position.

FIG. 1B is a top view of the SAID worn on the head of the animal in a supine position.

FIG. 2 is a longitudinal cross-sectional view of the SAID of FIG. 1A.

FIG. 3 is a lateral cross-sectional view of the SAID taken through a line 3-3 of FIG. 2.

FIG. 4 is an exploded perspective view of the SAID of FIG. 1A.

FIG. 5 is a longitudinal cross-sectional view of a body portion of the SAID of FIG. 1A.

FIG. 6 is a perspective view of the body portion of FIG. 4.

FIG. 7 is a perspective view of passageways and an interior collection chamber defined in the body portion that together form an airflow collection system illustrated with the animal's head.

FIG. 8 is a perspective view of a bite bar component of the SAID hooked onto the animal's incisors.

FIG. 9 is a perspective view of passageways defined in the body portion and the bite bar component that together form an anesthesia delivery system.

Like reference numerals have been used to identify like components in the figures.

### DETAILED DESCRIPTION OF THE INVENTION

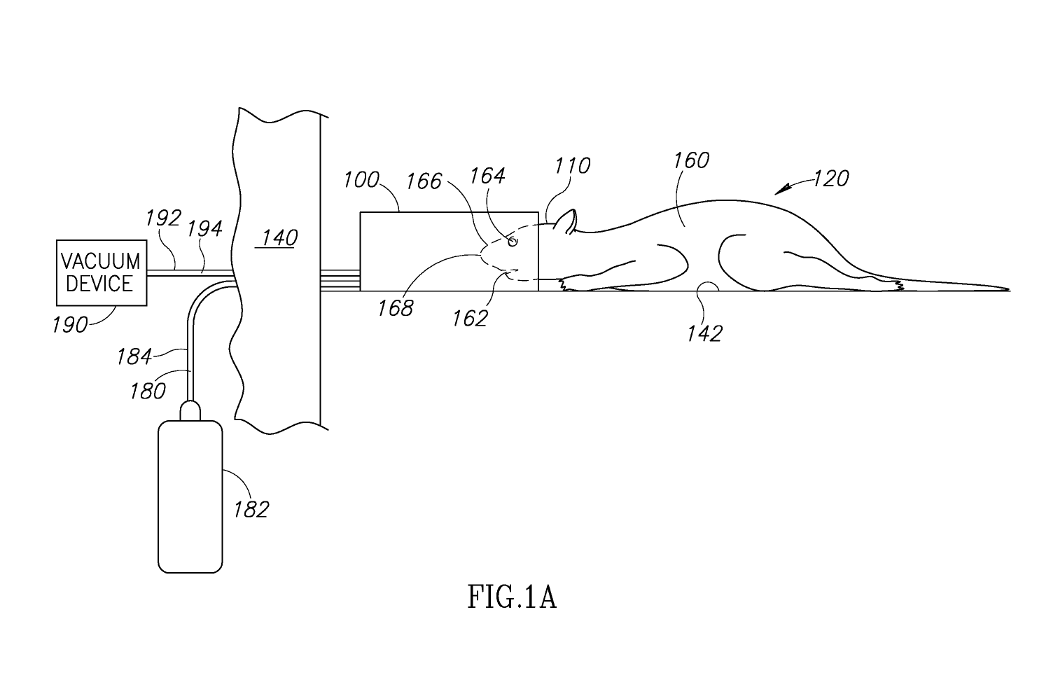

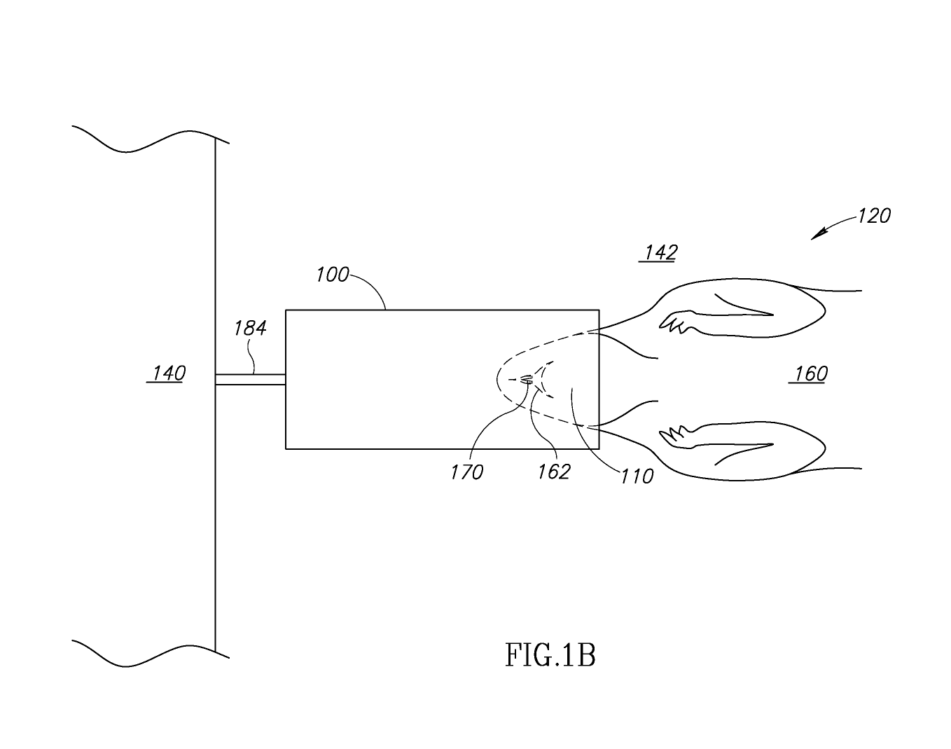

FIG. 1A is a side view of a Small Animal Imaging Device (“SAID”) 100 shown on a head 110 of an animal 120. In FIGS. 1A and 1B, the SAID 100 is illustrated before the animal 120 is imaged by an imaging device 140. By way of non-limiting examples, the imaging device 140 may be a magnetic resonance imaging (“MRI”) machine, a computerized tomography (“CT”) machine, a positron emission tomography (“PET”) machine, and the like. The imaging device 140 is configured to produce a scan of all or a portion of the animal 120 when the animal 120 is anesthetized. When being imaged by the imaging device 140, the animal 120 is positioned on a bed 142 in a desired imaging position (e.g., prone or supine). In FIG. 1A, the animal 120 is depicted in the prone position. However, this is not a requirement. The animal 120 may alternatively be positioned in a supine position (see FIG. 1B), on its side, and the like. The SAID 100 may be “machine-compatible” meaning the SAID 100 may be configured for use with the imaging device 140. In other words, the SAID 100 may be sized to fit into the imaging device 140 and constructed from one or more materials (e.g., polycarbonate) that will not show up in scans produced by the imaging device 140.

While the SAID 100 is illustrated in FIGS. 1A and 1B being used to image the animal 120, the SAID 100 may be used to anesthetize the animal 120 for other purposes. For example, the SAID 100 may be used to anesthetize the animal 120 for surgical procedures (e.g., a stereotaxic surgery), non-surgical procedures, and the like. Non-limiting examples of stereotaxic surgical procedures for which the SAID 100 may be used include craniotomies, tumor resections, and the like.

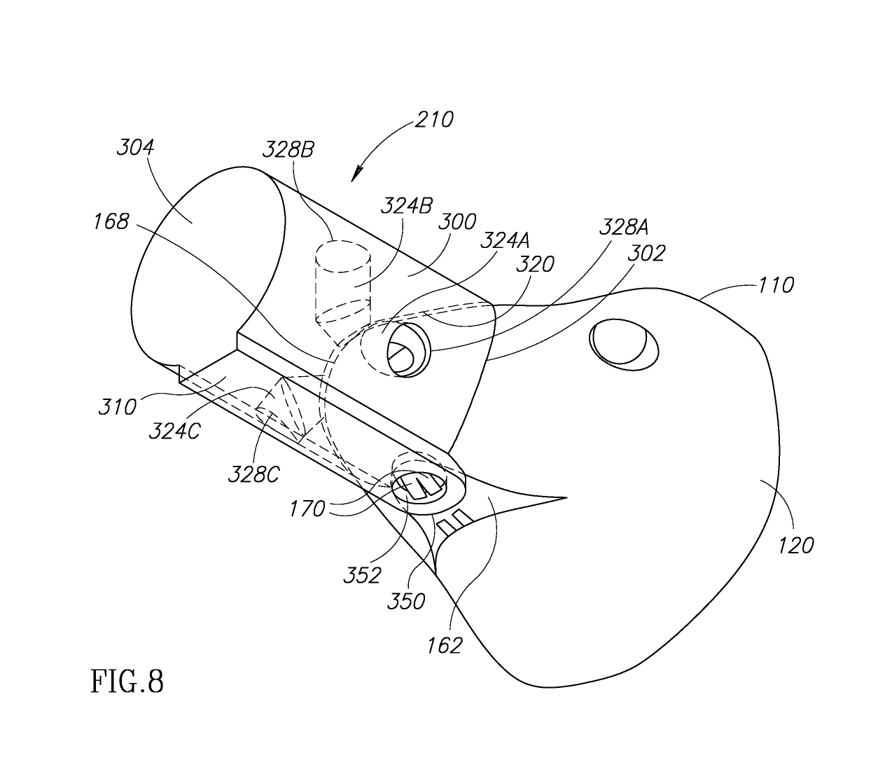

By way of non-limiting examples, the animal 120 may be a rodent (e.g., a mouse, a rat, and the like), a rabbit, and the like. The animal 120 has a body 160, a mouth 162, eyes 164, and a nose 166 with a tip 168. Referring to FIGS. 1B and 8, inside the mouth 162, the animal 120 has at least one tooth (e.g., incisors 170). Returning to FIG. 1A, while not illustrated, those of ordinary skill in the art appreciate that additional restraints may be used to help maintain the animal 120 in a substantially stationary position on the bed 142 when the animal 120 is being imaged by the imaging device 140.

The SAID 100 is an anesthesia mask configured to receive anesthesia gas(es) (illustrated as anesthesia 180) via a tube 184 from an anesthesia source 182 (e.g., a tank). The SAID 100 supplies the anesthesia 180 to the mouth 162 and/or the nose 166 of the animal 120. By way of a non-limiting example, the anesthesia 180 may be isoflurane, sevoflurane, halothane, and the like.

A vacuum device 190 is connected to the SAID 100 by a tube 192. A valve (not shown) may be positioned in line with the tube 192 or in between the tube 192 and the vacuum device 190. By way of another non-limiting example, an activated-carbon scavenging system (not shown) that absorbs the unused portions 194 may be positioned in line with the tube 192 or in between the tube 192 and the vacuum device 190. The vacuum device 190 is configured to draw unused portions 194 of the anesthesia 180 from the SAID 100 thereby preventing the unused portions 194 from exiting the SAID 100 and increasing laboratory safety.

By way of non-limiting examples, the tubes 184 and 192 may each be implemented as a vinyl tube (e.g., having a circular cross-sectional shape with approximately a three-millimeter outer diameter). The tubes 184 and 192 may be integrally formed with the SAID 100 or coupled thereto. Further, the tubes 184 and 192 may each be implemented as a single continuous tube or multiple tube sections connected together end-to-end.

The SAID 100 is configured to allow effortless positioning of the animal's head 110 and/or body 160, precise delivery of the anesthesia 180 to the animal 120, and collection of the unused portions 194 of the anesthesia 180. The SAID 100 may also help achieve artifact-free scans using the imaging device 140 because the SAID 100 encapsulates the animal's head 110.

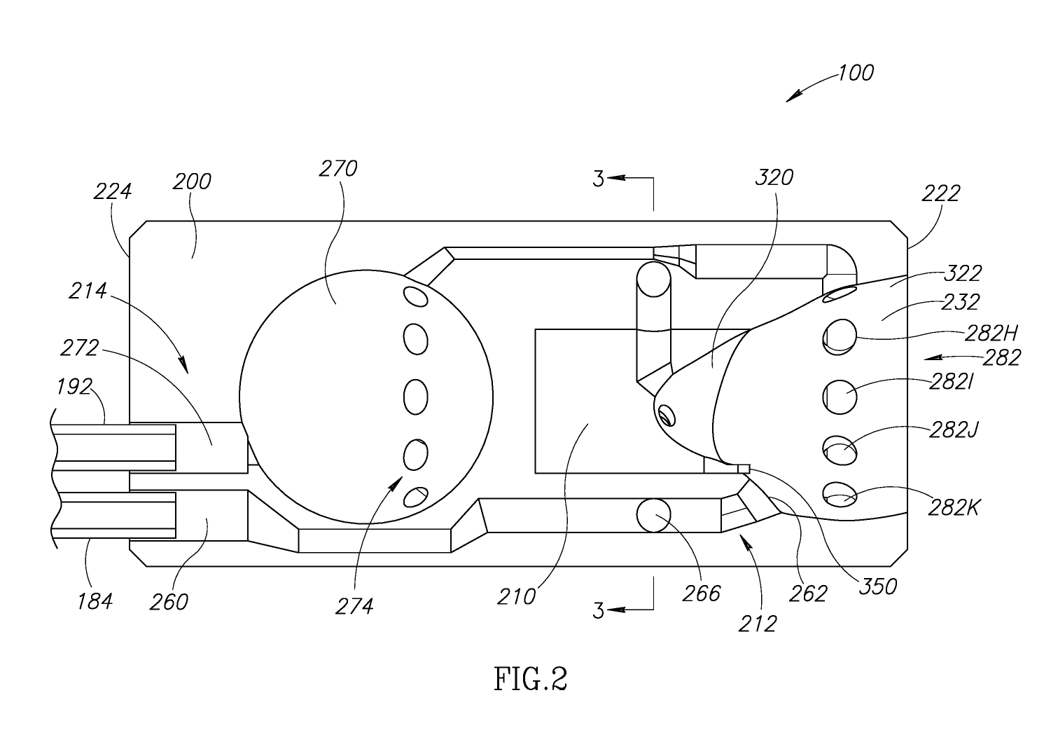

Referring to FIGS. 2-4, the SAID 100 may be characterized as including a body portion 200 and a removable bite bar component 210. The body portion 200 and the bite bar component 210 may each be printed using 3D printing technology. By way of an example, these components may be printed using a high-resolution stereolithographic printer capable of printing layers on the scale of tens of microns. Current 3D printing technology is capable of printing a wide variety of ultraviolet (“UV”) curable polymers. While clear or optically translucent UV-cured polycarbonate is typically used, any machine-compatible polymer may be used to construct the body portion 200 and/or the bite bar component 210.

Referring to FIG. 2, together, the body portion 200 and the bite bar component 210 form an anesthesia delivery system 212 that is connected by the tube 184 to the anesthesia source 182 (see FIG. 1A). The body portion 200 includes an airflow collection system 214 that is connected by the tube 192 to the vacuum device 190 (see FIG. 1A).

### Body Portion

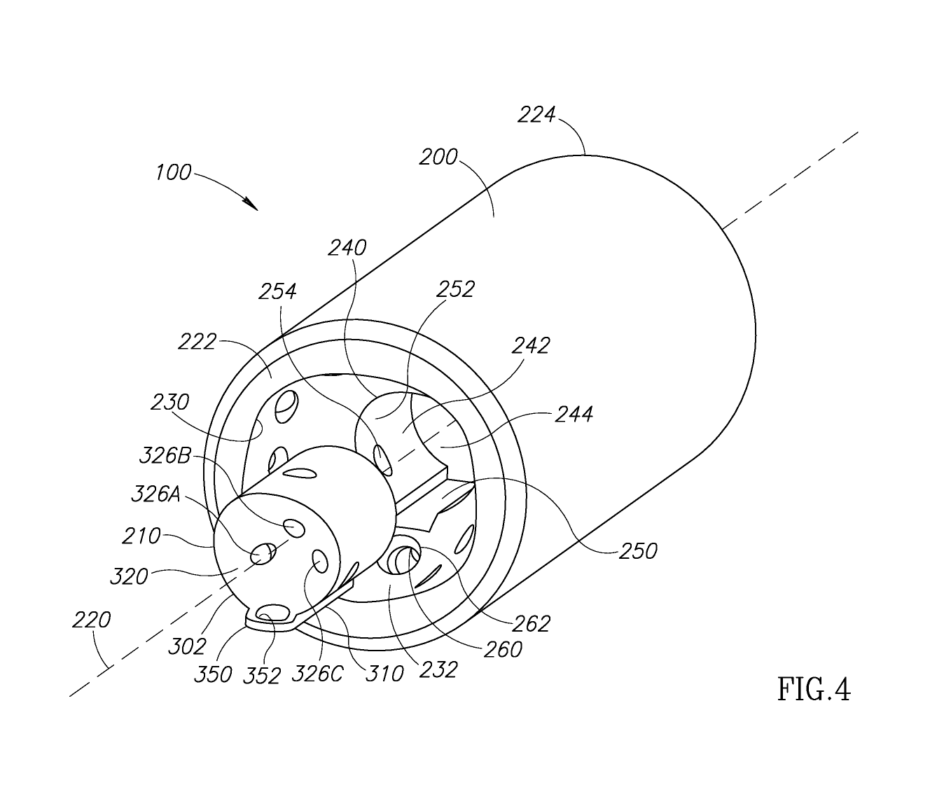

Referring to FIG. 4, the body portion 200 may have a generally cylindrical outer shape that extends along a longitudinal axis 220. The body portion 200 has a first end 222 opposite a second end 224. An opening 230 into an internal contoured cavity 232 is formed in the first end 222. The opening 230 is configured to receive the animal's head 110 (see FIGS. 1A, 1B, and 7-9) into the contoured cavity 232. The contoured cavity 232 is configured to at least partially encapsulate the animal's head 110 (see FIGS. 1A, 1B, and 7-9). Referring to FIG. 1A, the contoured cavity 232 (see FIGS. 2 and 4-6) keeps the animal's head 110 stable when positioning the animal 120 and delivering the anesthesia 180 to the nose 166 and/or the mouth 162.

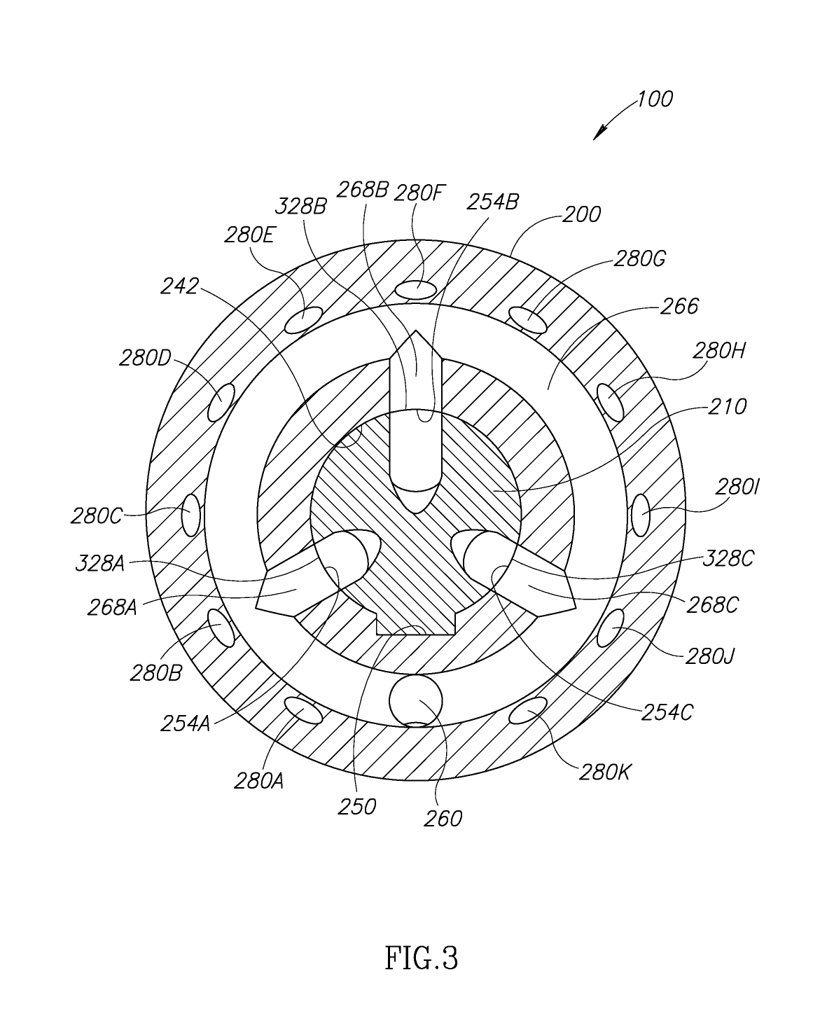

Referring to FIG. 4, the contoured cavity 232 has a rear opening 240 into an internal chamber 242 configured to receive the bite bar component 210. The chamber 242 extends longitudinally into the body portion 200 and is terminated by a wall 244. In the embodiment illustrated, both the chamber 242 and the bite bar component 210 are generally cylindrically shaped. A longitudinally extending slot 250 is formed in a sidewall 252 of the chamber 242. One or more outlets 254 (e.g., through-holes) are formed in the sidewall 252. Referring to FIG. 3, in the embodiment illustrated, the outlets 254 include three outlets 254A-254C arranged along the circumference of the chamber 242, and opening laterally into the chamber 242.

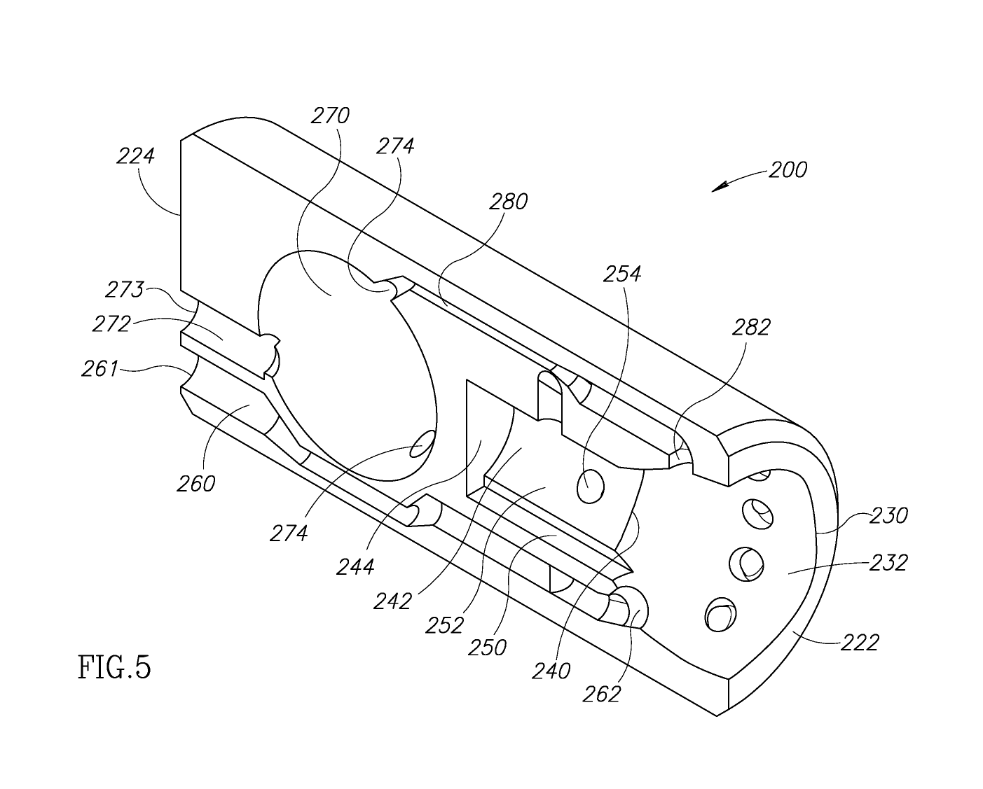

Referring to FIG. 5, the body portion 200 includes an open-ended channel 260 that extends from the second end 224 to the contoured cavity 232. At the second end 224, the channel 260 has an opening 261 configured to receive the tube 184 (see FIGS. 1A-2). By way of a non-limiting example, the opening 261 may have a circular cross-sectional shape with approximately a three-millimeter diameter.

The channel 260 has an opening 262 formed in the contoured cavity 232 at or near a location whereat the animal's mouth 162 (see FIGS. 1A, 1B, and 8) is positioned inside the contoured cavity 232. Thus, the opening 262 is in communication with the animal's mouth 162 (see FIGS. 1A, 1B, and 8) and provides the anesthesia 180 (see FIG. 1A) for inhalation by the animal 120 (see FIGS. 1A, 1B, and 7-9) via its mouth 162 (see FIGS. 1A, 1B, and 8). Referring to FIG. 3, in the embodiment illustrated, the channel 260 includes a ring-shaped portion 266 positioned longitudinally between the wall 244 (see FIGS. 4 and 5) and the rear opening 240 (see FIGS. 4 and 5) in the contoured cavity 232 (see FIGS. 2 and 4-6). The ring-shaped portion 266 is spaced apart laterally from and surrounds the chamber 242. The ring-shaped portion 266 is connected to each of the outlets 254A-254C by channel sections 268A-268C, respectively.

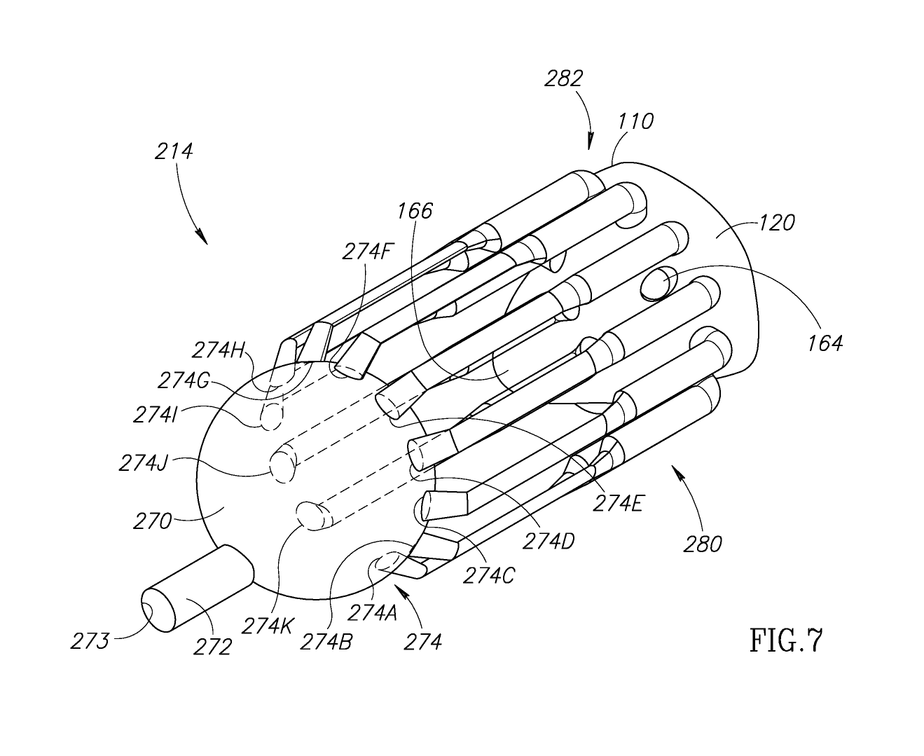

Referring to FIG. 5, the body portion 200 includes an interior collection chamber 270 positioned longitudinally in front of the animal's nose 166 (see FIGS. 1A, 7, and 9) between the second end 224 and the wall 244. In the embodiment illustrated, the collection chamber 270 has a generally spherical shape and has been implemented as a spherical void. However, this is not a requirement. An open-ended channel 272 extends from the second end 224 into the collection chamber 270. At the second end 224, the channel 272 has an opening 273 configured to receive the tube 192 (see FIGS. 1A and 2). By way of a non-limiting example, the opening 273 may have a circular cross-sectional shape with approximately a three-millimeter diameter. Referring to FIG. 2, the collection chamber 270 includes one or more openings 274 (e.g., through-holes). Referring to FIG. 7, in the embodiment illustrated, the openings 274 (see FIGS. 2, 5, and 7) include eleven substantially identical openings 274A-274K.

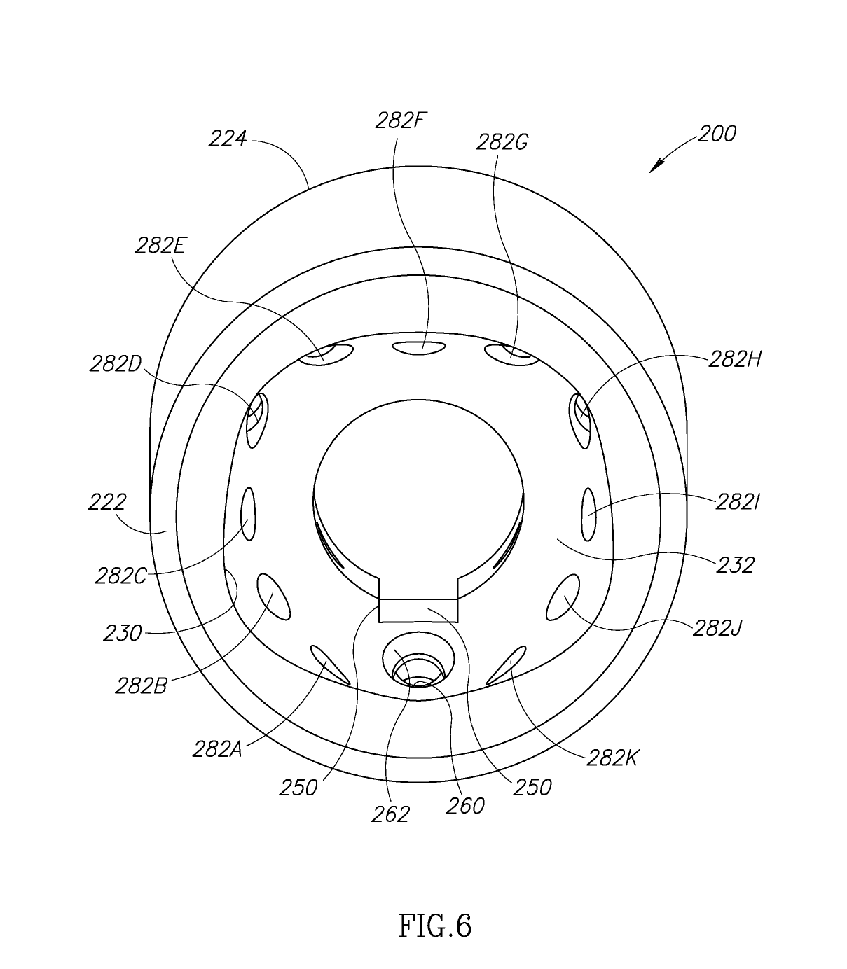

The openings 274 are each connected to a different one of a plurality of channels 280. Thus, referring to FIG. 3, in the embodiment illustrated, the channels 280 (see FIGS. 5 and 7) include eleven substantially identical channels 280A-280K. In the embodiment illustrated, the channels 280A-280K are spaced apart from and arranged circumferentially about the ring-shaped portion 266 and the chamber 242. The channels 280A-280K do not intersect with either the ring-shaped portion 266 or the chamber 242. Referring to FIG. 5, the channels 280 extend from the collection chamber 270 to inlets 282 formed in the contoured cavity 232. Each of the channels 280 is connected to a different one of the inlets 282. Thus, referring to FIG. 6, in the embodiment illustrated, the channels 280A-280K (see FIG. 3) extend from the collection chamber 270 (see FIGS. 2, 5, and 7) to inlets 282A-282K, respectively. As may be seen in FIG. 2, the inlets 282 are spaced apart from the opening 262 and are closer to the first end 222 than the opening 262. Referring to FIG. 7, the inlets 282 are positioned to be alongside the animal's head 110 (e.g., near or behind its eyes 164). In the embodiment illustrated, the inlets 282 are arranged in a circle that extends around the head 110. As shown in FIG. 7, together, the collection chamber 270, the channel 272, the openings 274, the channels 280, and the inlets 282 form the airflow collection system 214.

### Bite Bar Component

Referring to FIG. 8, as mentioned above, the bite bar component 210 has a generally cylindrical outer shape, which is defined by an outside surface 300. The bite bar component 210 has a first end 302 opposite a second end 304. Referring to FIG. 4, the bite bar component 210 includes a laterally outwardly extending rail or projection 310 configured to be slidably received longitudinally inside the slot 250. Referring to FIG. 8, in the embodiment illustrated, the projection 310 extends longitudinally between the first and second ends 302 and 304 of the bite bar component 210.

Referring to FIG. 4, the bite bar component 210 has a contoured recess 320 formed in the first end 302. The contoured recess 320 is configured to receive the animal's nose 166 (see FIGS. 1A, 7, and 9). Optionally, the contoured recess 320 is sized and shaped to provide a gap (not shown) between the bite bar component 210 and the animal's nose 166 (see FIGS. 1A, 7, and 9).

Referring to FIG. 2, together, the contoured cavity 232 and the contoured recess 320 define a discontinuous contoured surface 322 that may be intricately head-shaped and follow the natural contours of the animal's head 110 (see FIGS. 1A, 1B, and 7-9). In other words, the contoured surface 322 follows the outer shape of the portion of the animal's head 110 (see FIGS. 1A, 1B, and 7-9) positioned inside the contoured cavity 232 and the contoured recess 320. For example, the interior space defined by the contoured surface 322 is narrower near the animal's nose 166 (see FIGS. 1A, 7, and 9) than near the animal's eyes 164 (see FIGS. 1A and 7). Thus, together the contoured cavity 232 and the contoured recess 320 are neither cylindrical nor conical in shape. Instead, together the contoured cavity 232 and the contoured recess 320 may be characterized as being head shaped. Further, as shown in FIG. 2, neither the contoured cavity 232 nor the contoured recess 320 is rotationally symmetric about the longitudinal axis 220 (see FIG. 4). In other words, the contoured surface 322 is rotationally asymmetric about the longitudinal axis 220 (see FIG. 4). Thus, the animal's head 110 (see FIGS. 1A, 1B, and 7-9) fits within the contoured cavity 232 in only one orientation with respect to the body portion 200. Inside the SAID 100, the contoured recess 320 centers the tip 168 (see FIGS. 1A and 8) of the animal's nose 166 (see FIGS. 1A, 7, and 9) laterally.

The contoured surface 322 may be configured to fit the head 110 sufficiently to ensure external forces (e.g., vibrations) cannot easily dislodge the head 110 from the bite bar component 210. Additionally, the contoured surface 322 is configured to receive any individual animal of a particular type (e.g., any brown mouse). Even if the animal 120 gains or loses weight, the overall shape and size of the animal's head 110 remains relatively constant.

The contoured surface 322 may be designed using coordinates extracted from computer imaging data (e.g., MRI scans) that has been meshed together and edited (e.g., using one or more computer-aided design (“CAD”) programs). Such computer imaging data may be collected using the imaging device 140 or a similar machine. Geometry created in this manner reflects the shape of a particular animal's head and may be used to create the contoured surface 322. The contoured surface 322 may be configured for use with different animals. For example, the contoured surface 322 illustrated fits brown mice well. A similar contoured surface may be constructed for a different animal (e.g., a different type of mouse, a larger rodent, a rabbit, etc.) using the aforementioned design protocol and computer imaging data (e.g., MRI scans). Thus, the contoured surface 322 may be easily adapted to follow the contours of the heads of different types and sizes of animals. The anesthesia delivery system 212 and the airflow collection system 214 may be similarly adapted for use with different animals.

Referring to FIG. 8, one or more through-channels 324A-324C are defined in the bite bar component 210. The through-channels 324A-324C extend from outlets 326A-326C (see FIG. 4), respectively, formed in the contoured recess 320 (see FIGS. 2, 4, and 8) to inlets 328A-328C, respectively, formed in the outside surface 300. Referring to FIG. 3, the inlets 328A-328C are positioned to align with the outlets 254A-254C, respectively, when the bite bar component 210 is received inside the body portion 200. Referring to FIG. 8, in the embodiment illustrated, portions of the through-channels 324A-324C are tapered and narrow toward the outlets 326A-326C (see FIG. 4), respectively. Referring to FIG. 4, the tapered portions extend toward the contoured recess 320 and the animal's nose 166 (see FIGS. 1A, 7, and 9) at an angle with respect to the longitudinal axis 220. The outlets 326A-326C are formed in the contoured recess 320 at or near a location whereat the animal's nose 166 (see FIGS. 1A, 7, and 9) is positioned. Thus, the outlets 326A-326C are in direct communication with the tip 168 (see FIGS. 1A and 8) of the animal's nose 166 (see FIGS. 1A, 7, and 9) and provide the anesthesia 180 (see FIG. 1A) for inhalation by the animal 120 (see FIGS. 1A, 1B, and 7-9) via its nostrils (not shown).

Referring to FIG. 8, a bite member 350 extends longitudinally from the first end 302 and is positioned to contact one or more of the animal's teeth (e.g., the incisors 170). Thus, referring to FIG. 2, the bite member 350 extends into the contoured cavity 232 when the bite bar component 210 is positioned inside the body portion 200. Referring to FIG. 4, in the embodiment illustrated, the bite member 350 extends toward the animal's mouth 162 (see FIGS. 1A, 1B, and 8) from the projection 310. Referring to FIG. 8, the bite member 350 may include a laterally extending recess or through-hole 352 configured to receive and retain at least one of the animal's teeth (e.g., the incisors 170). In other words, the animal's tooth/teeth (e.g., the incisors 170) hook onto the through-hole 352 of the bite member 350. Inside the contoured cavity 232 (see FIGS. 2 and 4-6), the bite bar component 210 hooks around the animal's tooth/teeth (e.g., the incisors 170) and surrounds the tip 168 (see FIGS. 1A and 8) of the animal's nose 166 (see FIGS. 1A, 7, and 9).

Referring to FIG. 4, the projection 310 of the bite bar component 210 and the slot 250 the body portion 200 help assure proper alignment of the bite bar component 210 with the body portion 200 for easy setup. The bite bar component 210 is aligned with the body portion 200 by sliding the projection 310 inside the slot 250. The bite bar component 210 may be press-fit or friction fit into the chamber 242 of the body portion 200. Optionally, one or more fasteners (not shown) may be used to secure the bite bar component 210 to the body portion 200. By way of non-limiting examples, each of the fasteners (not shown) may be implemented using two opposing pieces of 3M™ Dual Lock™ Reclosable Fasteners that will not wear down from repeated use.

### Anesthesia Delivery System

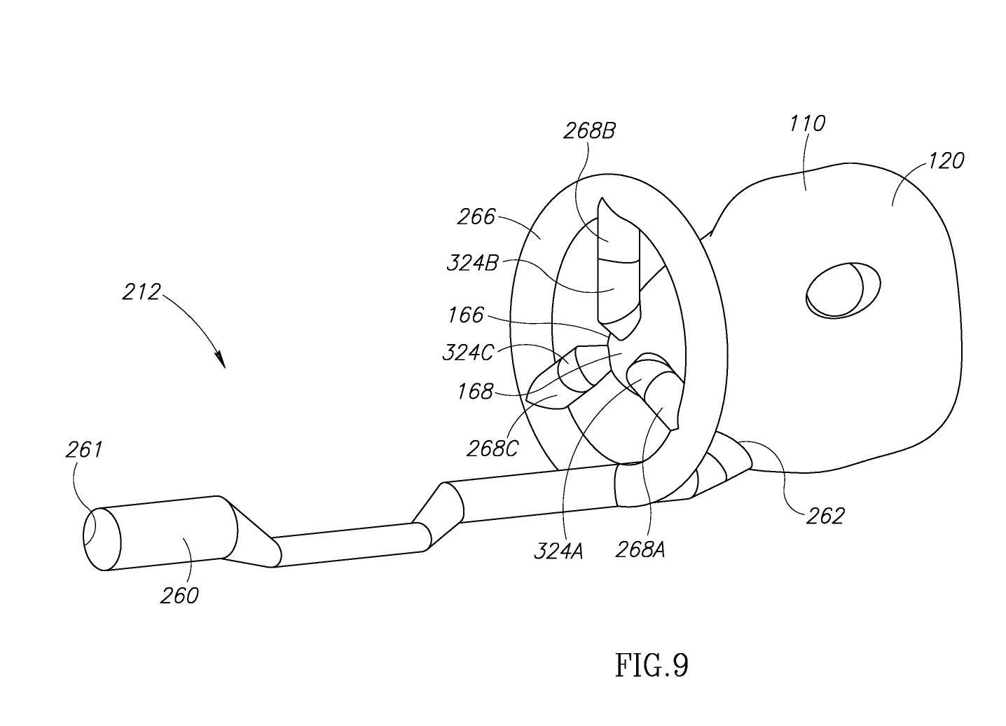

Referring to FIG. 2, as mentioned above, together, the body portion 200 and the bite bar component 210 form the anesthesia delivery system 212, which delivers the anesthesia 180 (see FIG. 1A) to the animal's nose 166 (see FIGS. 1A, 7, and 9) and/or the animal's mouth 162 (see FIGS. 1A, 1B, and 8). FIG. 9 depicts the passageways that form the anesthesia delivery system 212. As shown in FIG. 9, the anesthesia delivery system 212 includes the channel 260, the ring-shaped portion 266, the channel sections 268A-268C, the outlets 254A-254C (see FIG. 3), the inlets 328A-328C (see FIGS. 3 and 8), the through-channels 324A-324C, and the outlets 326A-326C (see FIG. 4).

Referring to FIG. 1A, the anesthesia 180 travels from the anesthesia source 182 through the tube 184 and into the channel 260 (see FIGS. 2-6 and 9). Referring to FIG. 9, a first portion of the anesthesia 180 (see FIG. 1A) travels through the channel 260 and enters the ring-shaped portion 266. The first portion travels through the ring-shaped portion 266 and exits therefrom through the channel sections 268A-268C. Referring to FIG. 3, the first portion travels through the channel sections 268A-268C and enters the inlets 328A-328C, respectively, of the bite bar component 210 through the outlets 254A-254C, respectively. Finally, referring to FIG. 4, the first portion of the anesthesia 180 (see FIG. 1A) travels through the through-channels 324A-324C (see FIGS. 8 and 9) and exits therefrom through the outlets 326A-326C, respectively, and enters the contoured recess 320 whereat the first portion may be inhaled by the animal 120 (see FIGS. 1A, 1B, and 7-9) through its nose 166 (see FIGS. 1A, 7, and 9). Referring to FIG. 2, at the same time, a second portion of the anesthesia 180 (see FIG. 1A) travels through the channel 260 bypasses the ring-shaped portion 266 and exits the channel 260 (through the opening 262) into the contoured cavity 232 whereat the second portion of the anesthesia 180 may be inhaled by the animal 120 (see FIGS. 1A, 1B, and 7-9) through its mouth 162 (see FIGS. 1A, 1B, and 8).

### Airflow Collection System

As mentioned above, referring to FIG. 7, together, the collection chamber 270, the channel 272, the openings 274, the channels 280, and the inlets 282 form the airflow collection system 214. Referring to FIG. 1A, as the anesthesia delivery system 212 (see FIGS. 2 and 9) is delivering the anesthesia 180 to the nose 166 and/or the mouth 162 of the animal 120, the airflow collection system 214 (see FIGS. 2 and 7) is collecting the unused portion 194 of the anesthesia 180 and helping to induce a steady flow of the anesthesia 180 from the outlets 326A-326C (see FIG. 4) and/or the opening 262 (see FIGS. 2, 4-6, and 9) to the inlets 282 (see FIGS. 2 and 7).

Referring to FIG. 7, suction provided by the vacuum device 190 (see FIG. 1A), causes the unused portion 194 (see FIG. 1A) of the anesthesia 180 (see FIG. 1A) to enter the inlets 282 and flow through the channels 280 toward the collection chamber 270. The unused portion 194 (see FIG. 1A) exits the channels 280 through the openings 274 and enters the collection chamber 270. The suction provided by the vacuum device 190 (see FIG. 1A) draws the unused portion 194 (see FIG. 1A) out of the collection chamber 270 via the channel 272 and into the tube 192 (see FIGS. 1A and 2).

The collection chamber 270 of the airflow collection system 214 helps ensures the channels 280 are equally depressurized so that a lower pressure or depressurized area is created by the inlets 282 near or behind the animal's eyes 164. Thus, the inlets 282 may each be at an equal (depressurized) pressure. Referring to FIG. 1A, the depressurized area induces a stream of the anesthesia 180 from primary anesthesia delivery points at the nose 166 (the outlets 326A-326C illustrated in FIG. 4) and the mouth 162 (the opening 262 illustrated in FIGS. 2, 4-6, and 9). The stream fills any spaces or gaps (not shown) between the animal's head 110 and the contoured surface 322 (see FIG. 2). The unused portion 194 of the anesthesia 180 is drawn out of the contoured cavity 232 (see FIGS. 2 and 4-6) by the inlets 282 (see FIGS. 2 and 7) and expelled from the SAID 100 by the external vacuum device 190.

### Operation

Referring to FIG. 1A, the SAID 100 may be easier to use than existing products on the market. First, the animal 120 is anesthetized in a gas chamber (not shown). Referring to FIG. 8, when the animal 120 is unconscious, the bite member 350 of the bite bar component 210 is hooked onto the animal's tooth/teeth (e.g., the incisors 170) with the animal's nose 166 (see FIGS. 1A, 7, and 9) positioned inside the contoured recess 320. The contoured recess 320 may be positioned such that the animal's tooth/teeth (e.g., the incisors 170) will automatically hook into the through-hole 352 of the bite member 350 when the animal's nose 166 (see FIGS. 1A, 7, and 9) is positioned inside the contoured recess 320. Next, referring to FIG. 4, the projection 310 of the bite bar component 210 (with the animal 120 attached thereto) is aligned with the slot 250 of the body portion 200. Then, the bite bar component 210 (with the animal 120 attached thereto) is slid into the chamber 242 of the body portion 200. As mentioned above, the bite bar component 210 may be press or friction fit into the chamber 242. Optionally, fasteners (not shown) may be used to secure the bite bar component 210 to the body portion 200. Referring to FIG. 2, if necessary, the tubes 184 and 192 may be connected to the channels 260 and 272, respectively, and/or the anesthesia source 182 and the vacuum device 190, respectively, at any time before the user initiates the anesthesia source 182 and the vacuum device 190. Referring to FIG. 1A, at this point in the protocol, the user initiates the vacuum device 190 and the anesthesia source 182 to provide continued anesthetization to the animal 120.

When the SAID 100 is used to image the animal 120, the SAID 100 and the animal 120 are positioned on the bed 142 in the desired position. The bed 142 (with the SAID 100 and the animal 120 thereupon) is positioned inside the imaging device 140 and imaging data is captured. Optionally, the SAID 100 and the animal 120 may be positioned within a core (not shown) that is inserted into the imaging device 140. However, as is appreciated by those of ordinary skill in the art, this portion of the protocol may differ for different types of imaging machines.

When anesthetization is no longer needed (after the imaging data has been collected or a procedure has been completed), the user turns off the vacuum device 190 and the anesthesia source 182. FIG. 2, in some embodiments, the tubes 184 and 192 may be disconnected from the channels 260 and 272, respectively, and/or the anesthesia source 182 and the vacuum device 190, respectively, at any time after which anesthetization is no longer needed. Then, the animal 120 is removed from the SAID 100. When the SAID 100 has been used to image the animal 120, the bed 142 (with the SAID 100 and the animal 120 thereupon) is removed from the imaging device 140 and the SAID 100 and the animal 120 are lifted from the bed 142. If used, the fasteners (not shown) are removed. Next, the bite bar component 210 (with the animal 120 attached thereto) is removed from the body portion 200. Then, the bite member 350 of the bite bar component 210 is removed from the animal's mouth 162.

The foregoing described embodiments depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

The invention claimed is:

1. An anesthesia mask configured to be worn on an animal's head that comprises a nose and a mouth with at least one tooth, the anesthesia mask being configured to provide anesthesia to at least one of the nose and the mouth, the anesthesia mask comprising:
a contoured surface defining a cavity configured to at least partially receive the animal's head, the contoured surface substantially following contours of a portion of the animal's head that comprises the nose and the mouth;
a bite member extending into the cavity, the bite member being configured to hook onto the at least one tooth;
at least one outlet formed in the contoured surface, the at least one outlet being in communication with at least one of the nose and the mouth;
a first opening configured to receive the anesthesia; and
at least one channel extending between the first opening and the at least one outlet, the at least one channel conducting the anesthesia from the first opening to the at least one outlet.

2. The anesthesia mask of claim 1, wherein the anesthesia mask extends along a longitudinal axis, and
the contoured surface is rotationally asymmetric about the longitudinal axis.

3. The anesthesia mask of claim 2, further comprising:
a first end; and
a second end opposite the first end along the longitudinal axis, the cavity having a cavity opening at the first end configured to receive the animal's head into the cavity, the first opening being positioned at the second end.

4. The anesthesia mask of claim 1, wherein the contoured surface helps prevent external forces from dislodging the at least one tooth from the bite member.

5. The anesthesia mask of claim 1 for use with a vacuum device, the anesthesia mask further comprising:
at least one inlet formed in the contoured surface;
a second opening connectable to the vacuum device; and
at least one passageway extending between the second opening and the at least one inlet, the at least one passageway being configured to conduct an unused portion of the anesthesia from the at least one inlet to the second opening.

6. The anesthesia mask of claim 5, wherein the at least one passageway comprises a collection chamber,
the at least one inlet comprises a plurality of inlets, and
the collection chamber is configured to substantially equalize pressure at each of the plurality of inlets.

7. The anesthesia mask of claim 1, further comprising:
a body portion comprising the first opening and a first portion of the contoured surface defining the cavity; and
a bite bar component removable from the body portion, the bite bar component comprising the bite member and a second portion of the contoured surface defining the cavity, the second portion being configured to receive the nose, the at least one outlet comprising a nose outlet formed in the second portion, the at least one channel comprising a first portion defined in the body portion and a second portion defined in the bite bar component.

8. The anesthesia mask of claim 7, wherein the body portion comprises a slot, and
the bite bar component comprises a projection configured to be slidably received inside the slot, engagement between the projection and the slot properly orienting the bite bar component with respect to the body portion.

9. The anesthesia mask of claim 1, wherein the at least one outlet comprises a nose outlet in communication with the nose and a mouth outlet in communication with the mouth, and the anesthesia mask further comprises:
a body portion comprising the first opening and the mouth outlet formed in a first portion of the contoured surface defining the cavity; and
a bite bar component removable from the body portion, the bite bar component comprising the bite member and the nose outlet formed in a second portion of the contoured surface defining the cavity, the at least one channel comprising at least one first passageway from the first opening to the nose outlet and at least one second passageway between the first opening and the mouth outlet.

10. The anesthesia mask of claim 9, wherein the body portion comprises a slot, and
the bite bar component comprises a projection configured to be slidably received inside the slot, engagement between the projection and the slot properly orienting the bite bar component with respect to the body portion.

11. The anesthesia mask of claim 1, wherein the bite member comprises a recess or through-hole configured to receive the at least one tooth.

12. An anesthesia mask for use with a vacuum device, the anesthesia mask to be worn on an animal's head that comprises a nose and a mouth, the anesthesia mask comprising:
a first opening configured to receive the animal's head into an internal cavity;
at least one outlet formed in the internal cavity, the at least one outlet being in communication with at least one of the nose and the mouth;
a second opening configured to receive anesthesia;
at least one channel extending between the second opening and the at least one outlet, the at least one channel providing the anesthesia from the second opening to the at least one outlet;
at least one inlet formed in the internal cavity and spaced apart from the at least one outlet;
a third opening connectable to the vacuum device; and
at least one passageway extending between the third opening and the at least one inlet, the at least one passageway being configured to conduct an unused portion of the anesthesia from the at least one inlet to the third opening.

13. The anesthesia mask of claim 12 for use with the mouth comprising at least one tooth, the anesthesia mask further comprising:
a bite member positioned inside the internal cavity, the bite member being configured to hook onto the at least one tooth.

14. The anesthesia mask of claim 13, wherein the bite member comprises a recess or through-hole configured to receive the at least one tooth.

15. The anesthesia mask of claim 12, wherein the internal cavity is defined by a rotationally asymmetric contoured surface configured to substantially follow contours of the animal's head.

16. The anesthesia mask of claim 12, wherein a stream of the anesthesia flows from the at least one outlet to the at least one inlet.

17. The anesthesia mask of claim 12, wherein the at least one passageway comprises a collection chamber,
the at least one inlet comprises a plurality of inlets,
the at least one passageway comprises a different passageway extending between the collection chamber and each of the plurality of inlets, and
the collection chamber is configured to substantially equalize pressure at each of the plurality of inlets.

18. The anesthesia mask of claim 12 for use with the mouth comprising at least one tooth, wherein the at least one outlet comprises a nose outlet in communication with the nose and a mouth outlet in communication with the mouth, and the anesthesia mask further comprises:
a body portion comprising the first opening, the second opening, the third opening, and the mouth outlet formed in a first portion of the internal cavity; and
a bite bar component removable from the body portion, the bite bar component comprising a bite member and the nose outlet formed in a second portion of the internal cavity, the bite member being positioned inside the first portion of the internal cavity and configured to hook onto the at least one tooth, the at least one channel comprising at least one first passageway from the first opening to the nose outlet and at least one second passageway between the first opening and the mouth outlet.

19. The anesthesia mask of claim 18, wherein the body portion comprises a slot, and
the bite bar component comprises a projection configured to be slidably received inside the slot, engagement between the projection and the slot properly orienting the bite bar component with respect to the body portion.

20. An anesthesia mask configured to be worn on a head of an animal, the head comprising a nose and a mouth, the anesthesia mask comprising:
a contoured surface defining a cavity configured to at least partially receive the animal's head, the contoured surface substantially following contours of a portion of the animal's head that comprises the nose and the mouth;
a nose outlet formed in the contoured surface, the nose outlet being in communication with the nose;
a mouth outlet formed in the contoured surface, the mouth outlet being in communication with the mouth, the mouth outlet being different from and spaced apart from the nose outlet;
an opening configured to receive anesthesia; and
at least one channel conducting the anesthesia from the opening to the nose and mouth outlets.

21. The anesthesia mask of claim 20, further comprising:
a body portion comprising the opening and a first portion of the contoured surface defining the cavity, the mouth outlet being formed in the first portion; and
a bite bar component removable from the body portion, the bite bar component comprising a bite member and a second portion of the contoured surface defining the cavity, the nose outlet being formed in the second portion, the at least one channel comprising at least one first passageway from the opening to the nose outlet and at least one second passageway from the opening and the mouth outlet, the bite member extending into the cavity and being configured to be hooked onto at least one tooth of the animal.
